(19) United States
(12) Patent Application Publication
(10) Pub. No.: US 2005/0182010 A1
(43) Pub. Date: Aug. 18, 2005

(54) Antiviral therapy on the basis of RNA interference

(76) Inventor: Petrus Theodorus de Haan, Oegstgeest (NL)

Correspondence Address: TRASK BRITT, P.O. BOX 2550, SALT LAKE CITY, UT, 84110, US

(21) Appl. No.: 10/964,598

(22) Filed: Oct. 12, 2004

Related U.S. Application Data
(63) Continuation of application No. PCT/NL03/00279, filed on Apr. 11, 2003.

(30) Foreign Application Priority Data
Apr. 12, 2002 (EP) 02076434.6

(51) Int. Cl. A61K 48/00; C12N 15/861

(52) U.S. Cl. 514/44; 435/456

(57) ABSTRACT

The invention concerns a gene therapy for treatment of animals and humans which suffer from an infection with a chronic virus such as HIV or HCV. It can also be used prophylactically to prevent chronic infection. The therapy makes use of a nucleotide construct stably integrated in the genome of the target cells of the virus, which is able to produce a single transcript or multiple transcripts capable of forming a double-stranded RNA which inhibits replication of the virus in situ.

### CROSS-REFERENCE TO RELATED APPLICATION

This application is a continuation of International Application No. PCT/NL03/00279, filed Apr. 11, 2003, designating the United States of America, corresponding to International Publication No. WO 03/087371 (published in English on Oct. 23, 2003), the contents of the entirety of which are incorporated by this reference.

### TECHNICAL FIELD

The invention relates generally to biotechnology and, more particularly, to a therapy against chronic viruses, such as HIV and Hepatitis viruses. In particular, the invention relates to a new gene technology-based therapy to inhibit virus replication and to prevent the formation of new virus particles.

### BACKGROUND OF THE INVENTION

Chronic viruses are viruses having long latency periods in infected individuals before disease symptoms develop. Chronic viruses include human immunodeficiency virus (HIV), Karposi's sarcoma-associated herpes virus (KSHV), Eppstein-Barr virus (EBV), Hepatitis B virus (HBV) and Hepatitis C virus (HCV). Especially HIV and HCV are major global health concerns. Patients infected with HIV will, at a certain point in time, lose immune response and the typical symptoms of the lethal acquired immune deficiency syndrome (AIDS) will become visible. HCV is the causal agent of chronic liver infections, which develop into lethal cirrhosis or liver cancer in a substantial percentage of infected individuals. To date, patients that carry these chronic viruses cannot be cured. The current anti-HIV therapies are based on the use of protease and reverse transcriptase inhibitors to prevent the formation of new virus particles by so-called Highly Active Anti-Retroviral Therapy (HAART). However, HAART is expensive and, therefore, not affordable by those countries that are the most affected. The inhibitors have many negative health effects and patients have to use the medication lifelong. HCV infections are currently treated with alpha-interferon alone or in combination with ribavirin, albeit with a low success rate. Additionally, the costs of both drugs are very high.

A major part of research is therefore dedicated to the development of vaccines. However, evidently due to the high mutation rates of the HIV and HCV genomes and, hence, the rapid emergence of virus variants that escape froth the above mentioned therapies, novel therapies are urgently needed.

HIV belongs to the lentivirus sub-family within the Retroviridae. Virus particles consist of two genomic single-stranded RNA molecules wrapped up with protein to nucleocapsids. Lipid envelopes wearing glycoprotein projections surround the nucleocapsids. Virions enter specific lymphoid cells by binding of gp120 to CD4 receptors. The maturational stages of all hemopoietic cells are distinguished by their expression of membrane molecules recognized by specific monoclonal antibodies (clusters of differentiation, CDs). Hence, the T-helper cells decorated with CD4 receptors are the main targets. However, a number of other cell types, such as monocytes, macrophages, dendritic cells, certain B-cells and hemopoietic stem cells (HSCs), also express some CD4 and, as a consequence, can be infected with HIV. Upon entrance, the nucleocapsids are released in the cytoplasm where the reverse transcriptase (included in the nucleocapsids) produces a cDNA copy of the genomic RNA. The reverse transcriptase and RNAseH, also included in the nucleocapsids, produce linear double-stranded DNA copies of the genomic RNA, which are targeted to the nucleus where they are integrated as proviral DNA into the nuclear DNA by a viral integrase, which is also included in the nucleocapsids.

The proviral genomes are flanked by long terminal repeats (LTRs). The 5′ LTR contains enhancer and promoter sequences essential for transcription of viral genes. The 3′ LTR contains the polyadenylation signals required for polyadenylation of the viral transcripts. The provirus contains three structural genes: gag, encoding the nucleocapsid proteins, pol, encoding the viral enzymes; and env, encoding the membrane glycoproteins. The mature proteins are formed after proteolytic cleavage of the primary translation products. In addition, the provirus has another five genes encoding regulatory proteins involved in virion infectivity, viral assembly and release, viral mRNA translocation and genome activation. The rev, tat and nef regulatory proteins are encoded by the early, multiple-spliced mRNA molecules; whereas, the structural proteins and other non-structural proteins are encoded by the late single-spliced (env, vif, vpr and vpu) and non-spliced (pol and gag) mRNA molecules.

The provirus is activated by antigen binding or by infection with other viruses. Provirus activation leads to expression of genes encoding the regulatory proteins, including the tat and rev proteins. Tat will further strengthen the 5′ LTR promoter, whereas rev will promote expression of the structural proteins and the viral enzymes. Viral nucleocapsids are formed in the cytoplasm and finally, virus particles will bud from the virus-infected CD4 cells. In this process, the virus kills T-helper cells, which have higher than threshold CD4 receptor levels. The other infected cell types remain in tact.

HIV infections, therefore, will, at a certain point in time, lead to a depletion of CD4+ T-lymphocytes and the typical AIDS symptoms will become visible. Uninfected CD4 T-lymphocytes lose their capacity to respond to new antigens. General antibody levels will decline. T-lymphocyte maturation in the thymus will be inhibited. Cytokine imbalances will occur leading to many immunological abnormalities. AIDS patients will finally die from progressive pathogen infections and exhaustion.

HCV belongs to the Flaviviridae family. Virus particles consist of a plus-stranded genomic RNA molecule wrapped up with C protein to nucleocapsids. Lipid envelopes with glycoprotein projections surround the nucleocapsids. The RNA molecule encodes a single large polyprotein, which is co- and post-translationally cleaved by viral and cellular proteases into ten different mature viral proteins, with the structural proteins (C, E1, E2 and p7) located in the amino-terminal part and the non-structural proteins (NS2, NS3, NS4a, NS4b, NS5a and NS5b) located at the carboxy-terminus. From a different reading frame other than the polyprotein overlapping with the C-encoding region, another non-structural protein (p16) is encoded. Virus particles have a tropism for hepatocytes and lymphoid cells. Upon entrance, the nucleocapsids are released in the cytoplasm and the genomic RNA molecules are translated directly. Virus replication takes place in the cytoplasm of infected cells. The majority of infected individuals becomes chronically infected and frequently shows chronic Hepatitis. The history of chronic HCV infections can vary dramatically between individuals. Some have minimal liver disease and never develop complications. Others develop severe and often lethal cirrhosis and/or hepatocellular carcinomas after many years of infection. Cirrhosis is momentarily the leading indication for liver transplantations. HCV is characterized by its presence at very low titers in the infected cells during the chronic phase and by its extremely high genomic variability. These characteristics significantly contribute to the lack of successful anti-HCV therapies.

Gene therapy has been considered as an attractive alternative anti-AIDS therapy (Romano et al., Stem Cells 17:191-202, 1999; Engel and Kohn, Front. Biosci. 4:26-33, 1999; Buchsacher and Wong-Staal, Human Gene Therapy 12:1013-1019, 2001). To date, gene therapy against diseases caused by other chronic viruses has not been suggested.

Two general approaches to gene therapy for HIV infection exist: to eliminate infected cells or to interfere with viral replication. The first approach includes attempts to increase the immune response in a patient directed against HIV and attempts to express toxins in HIV-infected cells. The second approach reported so far includes transgenic expression of dominant negative viral proteins (U.S. Pat. No. 5,908,923); targeted nucleases and ribonucleases (Singwi and Joshi, Front. Biosci. 5:556-579, 2001; Singwi et al., Gene Therapy 6:913-921, 1999); intracellular antibodies (Marasco et al., Human Gene Therapy 9:1627-1642, 1998; Mhasshilkar et al., Human Gene Therapy 10:1453-1467, 1999); interfering RNA molecules (Lamothe and Joshi, Front. Biosci. 5:527-555, 2000) such as sense decoy RNA molecules (Rosenzweig et al., J. of Virol. 71:2740-2746, 1997; Kohn et al., Blood 94:368-371, 1999), antisense RNA molecules (PCT International Publication WO 94/16066; DE 4225094; EP 0 386563; Veres et al., J. of Virology 72:1894-1901, 1998) and HIV-specific ribozymes (WO 94/26877; Rigden et al., Curr. Issues Mol. Biol. 2:61-69, 2000). To date, only in vitro experiments have been performed using HIV-infected human cell lines or CD34+38− cells isolated from patients' blood samples. It is now well established that gene expression in cells may be inhibited by the introduction into the cell of an RNA duplex (such as short RNAi) with a nucleotide sequence identical to a portion of the gene that expression is to be inhibited (e.g. WO 99/32619 and WO 99/53050). Such double-stranded RNA molecules may either be produced in vivo (i.e., in the cell) by introduction of a double-stranded RNA (dsDNA) into the cell (WO 00/63364) or may be derived from endogenous templates (WO 01/36646).

Comparable approaches have been used to confer virus resistance in other living systems such as plants. In plants, ribozymes, antisense and sense decoy RNAs confer virus resistance at relatively low levels and/or inefficiently, e.g., only a low percentage of the transformed cells (plants) show the desired phenotype. From these experiments, it can be deduced that the levels of inhibition of HIV replication conferred by these molecules is too low to fully keep HIV proviral DNA molecules in a latent state. Furthermore, although transfer of inhibitory double-stranded RNA products to daughter cells does occur, the inhibition of expression of specific genes by the above-described methods remains a transient phenomenon. In the case of infection with a chronic virus, the methods of the prior art do not, therefore, result in effective therapeutic treatment. The virion will eventually reappear or reactivate once inhibition diminishes since daughter cells are not effectively protected.

Thus, a need still exists for a more robust and efficient gene therapy for the inhibition of the replication of HIV and other chronic viruses including HCV.

### SUMMARY OF THE INVENTION

Provided is a method for the inhibition of replication of a chronic virus in cells of an animal or human by stably integrating a nucleotide construct into the genome of the target cell of the virus or a progenitor cell thereof capable of generating the target cell of the virus, which nucleotide construct is able to produce one transcript or multiple transcripts capable of forming a double-stranded RNA with nucleotide sequence homology to one or more nucleotide sequences of the virus, which are essential for replication of the virus. Thus, an embodiment of the invention also includes such a method wherein a progenitor cell (i.e., a hemopoietic stem cell, a lymphoid stem cell, or a T-helper cell (including a naive cell and a memory cell)) is provided with the nucleotide construct and wherein the progenitor cell is capable of generating the target cell of the virus. Preferably, the progenitor cells are stem cells.

In the specific case of HIV, the invention provides for a method for inhibition of human immunodeficiency virus replication comprising the steps of: a) isolating from a person cells selected from the group consisting of a hemopoietic stem cells, lymphoid stem cells and T-helper lymphocytes; b) stably integrating into the genome of the cells a nucleotide construct which is able to produce one transcript or multiple transcripts capable of forming a double-stranded RNA with nucleotide sequence homology to one or more nucleotide sequences of human immunodeficiency virus which are essential for replication; and c) re-introducing the cells in the person from whom they have been isolated.

Alternatively, a nucleotide construct which is able to produce one transcript, or multiple transcripts capable of forming a double-stranded RNA with nucleotide sequence homology to one or more nucleotide sequences of the human immunodeficiency virus that are essential for replication, is stably integrated in the progenitor cells by introducing the nucleotide construct in situ, without isolation and re-introduction of the cells from a person.

In the specific case of HCV, the invention provides for a method for inhibition of HCV replication comprising: a) isolating hepatocytes, liver stem cells, mesenchymal adult progenitor cells, mesenchymal stem cells or hemopoietic stem cells from a person; b) introducing into the cells a nucleotide construct that is able to produce one transcript or multiple transcripts capable of forming a double-stranded RNA with nucleotide sequence homology to one or more nucleotide sequences of the Hepatitis C virus, and c) re-introducing the cells in the liver of the person from whom they have been isolated.

Further, the invention provides an animal or human cell that is the target cell for a chronic virus characterized in that a nucleotide construct is introduced into it that is stably integrated into the genome of the cell and the nucleotide construct is able to produce one transcript or multiple transcripts capable of forming a double-stranded RNA with nucleotide sequence homology to one or more nucleotide sequences of the virus that are essential for replication.

In the alternative, an animal or human progenitor cell is provided that is able to generate a target cell for a chronic virus characterized in that into the progenitor cell a nucleotide construct is introduced that is stably integrated into the genome of the progenitor cell and that is able to produce one transcript or multiple transcripts capable of forming a double-stranded RNA with nucleotide sequence homology to one or more nucleotide sequences of the chronic virus that are essential for replication.

More specifically, such a target cell is a human hemopoietic stem cell or lymphoid stem cell or a T-helper lymphocyte (including a naive or memory cell) or such a target cell may be a hepatocyte or a liver stem cell, a mesenchymal adult progenitor cell or a mesenchymal stem cell, characterized in that a nucleotide construct is introduced into it that is stably integrated into the genome of the cell and that is able to produce one transcript or multiple transcripts capable of forming a dsRNA with nucleotide sequence homology to one or more nucleotide sequences of a human immunodeficiency virus that are essential for replication.

Also part of the invention is a nucleotide construct harboring a nucleotide sequence of at least 40 nucleotides with nucleotide sequence homology to a gene or part of a gene of a chronic virus that is able to infect an animal or human cell, wherein the nucleotide sequence is also present as an inverted repeat or wherein the nucleotide sequence is flanked by two promoters and the construct can be stably integrated into the genome of the cell and when transcribed, yields one transcript or multiple transcripts capable of forming a dsRNA from the sequence and its inverted repeat or from the sequence flanked by two promoters. Preferably, in such a nucleotide construct, the nucleotide sequence and its inverted repeat are separated by an intron.

Another embodiment of the invention is such a nucleotide construct that comprises multiple nucleotide sequences of at least 40 nucleotides long which are homologous to different parts of the same gene or to different genes or parts thereof of the virus, wherein the nucleotide construct is also present as an inverted repeat, or wherein the nucleotide construct is flanked by two promoters.

In another aspect, the present invention provides a method for the production of an animal or human cell or a progenitor cell thereof, that is the target cell for a chronic virus, the method comprising providing an animal or human cell or a progenitor cell thereof, and stably integrating into the genome of the provided cell a nucleotide construct that is able to produce one transcript or multiple transcripts capable of forming a double-stranded RNA with nucleotide sequence homology to one or more nucleotide sequences of the virus that are essential for replication.

In another aspect, the present invention provides a method of treating an infection of a chronic virus in cells of a subject in need thereof, the method comprising contacting the cells or progenitor cells thereof, capable of generating the cells of a patient suffering from the infection with a therapeutically effective amount of a reagent that stably integrates a nucleotide construct into the genome of the cells or the progenitor cells, the nucleotide construct is able to produce one transcript or multiple transcripts capable of forming a double-stranded RNA with nucleotide sequence homology to one or more nucleotide sequences of the virus that are essential for replication of the virus.

In one embodiment of this aspect, the invention provides such a method wherein the chronic virus is human immunodeficiency virus and wherein the cells are hemopoietic stem cells, lymphoid stem cells or T-helper lymphocytes.

In yet another embodiment of this aspect, the invention provides such a method wherein the chronic virus is Hepatitis C virus and wherein the cells are hepatocytes, liver stem cells, mesenchymal adult progenitor cells, mesenchymal stem cells or hemopoietic stem cells.

In one embodiment of a method of treating an infection of a chronic virus in cells of a subject in need thereof, the invention provides such a method wherein the reagent comprises a viral vector according to the invention.

A method of treating an infection of a chronic virus according to the present invention may be performed in various ways. For instance, prior to contacting the infected cells or progenitor cells thereof with the reagent, the cells or progenitor cells may be isolated from the patient and the thus transduced cells or progenitor cells are re-introduced into the patient.

### DETAILED DESCRIPTION OF THE INVENTION

The term “stem cell” as used herein refers to those cells that are the progenitors of all specialized cells in the body and include blood stem cells (hematopoietic cells) that reside in bone marrow and continuously produce a variety of blood and immune system cells, mesenchymal stem cells that are the source of new bone, cartilage, and connective tissue cells, and neuronal stem cells that produce a variety of nervous system tissue. The precursor to all these more specialized stem cells is the “pluripotential stem cell” (PSC) that divides by asymmetrical division (i.e., division results in one differentiated daughter cell and one undifferentiated stem cell). The more specialized stem cells include “multipotential stem cells,” lymphoid progenitors and myeloid progenitors. Lymphoid progenitors (stem cells) include the pre B-cell (resulting in B-lymphocytes, such as plasma cell and memory cell) and the pre T-cell (resulting in T-lymphocytes including the T-helper cell, the cytotoxic T-cells, the suppressor T-cells and probably also NK cells). Myeloid progenitors (stem cells) include GM stem cells (resulting in granulocytes, monocytes and macrophages), eosinophilic stem cells (resulting in eosinophilic granulocyte), megakaryo stem cells (platelets), and erythro stem cells (reticulocyte and erythrocyte). Other more specialized stem cells are those cells that result in differentiated daughter cells, such as endothelial progenitor cells, neural progenitor cells, oligodendrocyte progenitor cells, pancreatic progenitor cells, hematopoietic progenitor cells, glial progenitor cells or hepatocyte progenitor cells.

The term “progenitor cell” as used herein refers to those cells that are the ancestor in direct line of the daughter cells that result from cell division and include pluripotential and other stem cells but also some specialized cells. Thus, a progenitor cell of a T-lymphocyte may be, besides a stem cell, a naive T-lymphocyte or a memory T-lymphocyte or any T-lymphocyte or any cell that produces daughter T-lymphocytes.

The term “stable integration” or “stably integrated” as used herein is defined as the long-term or permanent incorporation of a nucleic acid construct into the genome of a host cell into which such a construct is introduced after being brought into contact therewith, e.g., by using vectors or other delivery systems as described hereinbelow. Such incorporation may be the result of homologous recombination and results in cells propagating the chromosomally incorporated construct to all daughter cells that result from cell division.

The term “nucleotide sequence homology” as used herein denotes the presence of homology between two polynucleotides. Polynucleotides have “homologous” sequences if the sequence of nucleotides in the two sequences is the same when aligned for maximum correspondence. Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence homology” for polynucleotides, such as 50, 60, 70, 80, 90, 95, 98, 99 or 100 percent sequence homology, may be determined by comparing two optimally aligned sequences over a comparison window, wherein the portion of the polynucleotide sequence in the comparison window may include additions or deletions (i.e. gaps) as compared to the reference sequence (which does not comprise additions or deletions) for optimal alignment of the two sequences. The percentage is calculated by: (a) determining the number of positions at which the identical nucleic acid base occurs in both sequences to yield the number of matched positions; (b) dividing the number of matched positions by the total number of positions in the window of comparison; and (c) multiplying the result by 100 to yield the percentage of sequence homology. Optimal alignment of sequences for comparison may be conducted by computerized implementations of known algorithms or by inspection. Readily available sequence comparison and multiple sequence alignment algorithms are, respectively, the Basic Local Alignment Search Tool (BLAST) (Altschul, S. F. et al. 1990, J. Mol. Biol. 215:403; Altschul, S. F. et al. 1997, Nucleic Acid Res. 25:3389-3402) and ClustalW programs both available on the internet. Other suitable programs include GAP, BESTFIT and FASTA in the Wisconsin Genetics Software Package (Genetics Computer Group (GCG), Madison, Wis., USA).

Inhibition of replication of a chronic virus is obtained by a method of producing sequence-specific inhibition of gene expression by introducing a nucleotide construct capable of forming, upon transcription, one transcript or multiple transcripts capable of forming a dsRNA. A process is provided for inhibition of expression of an homologous viral gene in a human or animal cell that is the target for the virus or that can generate cells that are the target cells for the virus. The process comprises introduction of nucleotide constructs comprising nucleotide sequences homologous to sequences of the virus are essential for replication that may form RNA molecules that are partially or fully double-stranded. When present in the target cells, such nucleotide constructs inhibit expression of the homologous viral gene(s) in a sequence-specific manner in a process referred to as RNA silencing (Ding S. W. 2000, Curr. Opin. Biotechnol. 11:152-156). Inhibition of gene expression refers to the absence (or observable decrease) in the level of protein and/or mRNA product from the homologous viral gene(s). Specificity refers to the ability to inhibit the target gene without manifest effects on other genes of the cell.

Generally, all genes of a virus are essential for that virus. However, inhibition through RNA silencing by dsRNA of a viral target gene is often not enough for inhibition of virus replication. If the virus is able to overcome, circumvent or overload the host RNA silencing before it requires the targeted gene, the virus replication is not inhibited. Thus, only a viral target gene that is required before the virus is able to overcome, circumvent or overload the host RNA silencing is considered essential for virus replication. Preferably, early viral genes are targeted in order to prevent virus replication.

A nucleotide construct that is able to form RNA containing a nucleotide sequence identical to a portion of the viral gene is preferred for inhibition. RNA sequences with insertions, deletions and single point mutations relative to the target viral sequence (i.e., not identical but homologous to the viral sequence) have also been found effective for inhibition. This is especially valuable in circumstances where the virus is prone to genomic variation. Even if the virus' genomic sequence shows variation, the inhibiting effect of the nucleotide construct pertains. Sequence identity may be optimized by sequence comparison and alignment algorithms known in the art (see Gribskov, M. and J. Devereux (Eds.), Sequence Analysis Primer, Stockton Press, New York, 1991, and references cited therein) and calculating the percent difference between the nucleotide sequences, for instance, by using the Smith-Waterman algorithm as implemented in the BESTFIT software program using default parameters (e.g., University of Wisconsin Computing Group). Greater than 90% sequence identity, or even 100% sequence identity, between the inhibitory nucleotide sequence and the portion of the target viral gene is preferred. Alternatively, the duplex or double-stranded region of the RNA transcript or transcripts encoded by the nucleotide construct may be defined functionally as a (double-stranded) nucleotide sequence that, upon denaturation, is capable of hybridizing with a portion of the target gene transcript (e.g., under conditions of 400 mM NaCl, 40 mM PIPES pH 6.4, 1 mM EDTA, at a temperature of 50° C. to 65° C. and hybridization for 12-16 hours, followed by washing). The length of the identical nucleotide sequences should be at least 40 nucleotides, but preferably larger, such as 50, 100, 200, 300, or 400 nucleotides. As disclosed herein, 100% sequence identity between the inhibiting nucleotide construct and the target endogenous gene is not required to practice the present invention and is difficult to obtain in the event that the target viral sequence changes or has changed due to genomic variation of the virus. Thus, the invention has the advantage of being able to tolerate sequence variations that might be expected due to genetic mutation, strain polymorphism or evolutionary divergence. Sequence homology between dsRNA as used in the present invention and one or more nucleotide sequences of the virus that are essential for replication of the virus and that are to be inhibited is suitably greater than 80%, preferably greater than 90%, more preferably, greater than 95%, even more preferably, greater than 98%, and most preferably, greater than 99%.

For transcription from an expression DNA construct (for transgenic in vivo transcription), a regulatory region, such as a promoter, enhancer, splice donor and acceptor, or polyadenylation) may be used to transcribe the DNA. The RNA strand(s) may or may not be polyadenylated; the RNA strand(s) may or may not be capable of being translated into a polypeptide by the cell's translational apparatus. Preferably, the constructs are not translated into a polypeptide. The use and production of an expression construct are known in the art (vide WO 97/32016; U.S. Pat. Nos. 5,593,874, 5,698,425, 5,712,135, 5,789,214 and 5,804,693).

A nucleotide construct according to the present invention is stably integrated into the chromosomal DNA of a target cell or progenitor thereof. Such stable integration may be obtained by one of several methods. Physical methods of introducing nucleic acids into cells may, for instance, be used. Such methods are referred to as naked DNA transfer and include injection of a solution containing the nucleotide construct, soaking the cells in a solution containing the nucleotide construct, or electroporation of cell membranes in the presence of the nucleotide construct. Other methods known in the art for naked DNA transfer may be used, such as lipid-mediated carrier transport, chemical-mediated transport, such as calcium phosphate and the like. Through naked DNA transfer, it is possible to insert the nucleotide construct in a specific place in the genome of the target cell or progenitor thereof. This is done by engineering the nucleotide construct in between parts which are homologous to, preferably non-coding, human sequences. This nucleotide sequence will then be inserted on a specific place in the genome through homologous recombination. By using this mechanism, it is possible to insert the nucleotide sequence at a spot that is highly transcribed and where it does not disturb normal cell functions. The larger the length and the homology of the flanking sequences, the higher the efficiency of site-directed homologous recombination.

An alternative delivery system may comprise the use of liposomes in which the nucleotide construct is captured or enveloped or cationic amphiphilic compounds to which the nucleotide construct is complexed.

A preferred method to introduce the nucleotide construct in the target cell or progenitor thereof comprises the use of a viral vector system. To that end, the nucleotide construct is engineered into a viral vector DNA molecule and packaged into a viral particle. The viral particle then accomplishes efficient introduction of the nucleotide construct into the target cell or progenitor thereof. Suitable viral vectors include such viruses as retroviruses, lentiviruses, adenoviruses, adeno-associated virus or herpes simplex virus, which are made replication deficient, i.e., by removal of viral genes essential for replication.

Stable integration into the host cell DNA is a natural characteristic of retroviruses, so that genes introduced by these vectors can be maintained for the life of the cell. Furthermore, the vector will be present in all daughter cells that result from cell division. Suitable retroviruses include murine leukemia virus (MLV) and moloney murine leukemia virus (Mo-MuLV). The advantages of using a virus such as MLV are that there is no pre-existing immunogenicity to such a virus, that there will be no immune response to viral gene products and that the virions are relatively easy to produce. However, the virus requires dividing cells for infection and may integrate randomly into the host cell genome leading to potential oncogenesis by insertional mutation. Also, it is known that the LTRs may interfere with gene expression.

Suitable lentiviruses include bovine lentiviruses, such as bovine immunodeficiency virus and Jembrana disease virus, equine lentiviruses such as equine infectious anemia virus, feline lentiviruses such as feline immunodeficiency virus (FIV), panther lentivirus and puma lentivirus, ovine/caprine lentiviruses such as Brazilian caprine lentivirus, caprine arthritis-encephalitis virus, caprine lentivirus, Maedi-Visna virus, ovine lentivirus and Visna lentivirus and the primate lentivirus group including such viruses as human immunodeficiency virus (HIV) and simian immunodeficiency virus (SIV). An advantage of the use of lentivirus is that no dividing cells are required and that they may thus be used for in vivo applications to transduce non-dividing cells (e.g., memory T-lymphocytes, hemopoietic stem cells, neurons).

Adeno-associated virus (AAV) vectors are advantageous because they both expeditiously infect non-dividing cells and undergo site-specific integration into the host cell genome, resulting in long-term transduction. Adeno-associated virus (AAV) is a non-pathogenic dependent parvovirus with a broad host range, capable of high levels of transduction and stable integration into the host cell genome.

All of the above-described delivery systems may be used in a method of the invention, provided that stable integration in the host cell genome is achieved.

Most preferred are the use of viral vectors derived from lentiviruses, adeno-associated virus (AAV), or combinations such as adenovirus-AAV hybrid vectors. The methods for constructing viral vectors and packaging those into a viral particle are known to a person skilled in the art.

The target cells to be transduced or transfected are preferably those cells which are the subject of viral infection. Of course, the virus that is used to transduce these cells must be able to infect these cells. Lentiviruses are known to infect cells bearing the CD4 receptor including T-lymphocytes and would be suitable to transduce the HIV target cells in situ. AAV type 2 is known to infect hepatocytes and thus are suitable to transduce liver cells in situ.

As an alternative, stem cells or progenitor cells that generate the cells that are the target for viral attack are used for transduction in a method of the invention. In the case of HIV, hemopoietic or lymphoid stem cells or memory or naive T-helper lymphocytes are suitable. In the case of HCV, liver stem cells, hemopoietic stem cells, mesenchymal stem cells, or mesenchymal adult progenitor cells are suitable.

Pluripotent hemopoietic stem cells are present in the hemopoietic tissues within the bone marrow of infants and adults. Approximately one out of 2000 cells is a stem cell. The stem cells divide and differentiate into either myeloid stem cells or lymphoid stem cells and from there, into mature blood and immune system cells.

The myeloid stem cells are the precursors of granulocytes, mast cells, dendritic cells, monocytes, macrophages, erythrocytes and megakaryocytes. The erythrocytes become red blood cells and the megakaryocytes turn into platelets. All together, the myeloid stem cells form the blood tissue and some cell types play a role in immunity. For example, the monocytes and macrophages play key roles in phagocytosis and antigen-presentation to lymphocytes. Granulocytes also show phagocytosis (neutrophils, eosinophils) or they release antimicrobial compounds (basophils). Mast cells also release pharmacologically active compounds at the site of infection. Dendritic cells play a key role in antigen presentation to T-lymphocytes.

The lymphoid stem cells differentiate into B-lymphocytes in the bone marrow. The B-lymphocytes are the antibody-producing cells and responsible for the humoral defense responses. In the bone marrow, B-cells undergo an orderly sequence of immunoglobulin gene arrangements that are antigen-independent. Mature naïve B-cells expressing membrane-bound IgM and IgD molecules leave the bone marrow and migrate to the secondary lymphoid organs. Interaction with antigen, either directly or via antigen-presenting cells, induces clonal selection of the affected B-cells in a process mediated by specific cytokines, secreted by activated T-lymphocytes (effector cells). B-cells divide and differentiate, generating populations of short-living plasma cells or long-living memory cells that secrete specific immunoglobulins.

Lymphoid stem cells also migrate from the bone marrow to the thymus, where they differentiate into T-lymphocytes and (natural killer) NK-lymphocytes. From the thymus, mature T-lymphocytes and NK cells are actively dispersed to the peripheral tissues. The T- and NK-lymphocytes are responsible for the cellular defense responses. T-lymphocytes recognize antigens that must be presented together with MHC molecules on the surface of antigen-presenting cells, cancer cells, virus-infected cells or grafts.

A sub-population of T-lymphocytes bearing CD4 recognize antigen associated with MHC class II molecules and generally function as T-helper cells. Naive T-helper lymphocytes are activated through antigen recognition and form memory and effector T-helper lymphocytes. Activation of T-helper lymphocytes leads to cell division and gives rise to clones of effector cells. The effector cells secrete specific sets of cytokines that play a central role in the activation of cells involved in specific responses, yielding memory, like mature B-lymphocytes or T-cytotoxic lymphocytes and other cells involved in non-specific responses like NK-lymphocytes, monocytes and macrophages.

T-lymphocytes bearing CD8 recognize antigen associated with MHC class I molecules and function as T-cytotoxic cells. After antigen-binding by T-cytotoxic lymphocytes, cytokines like IL-2 produced by T-helper lymphocytes induce proliferation and differentiation of the T-cytotoxic lymphocytes into cytotoxic T-lymphocytes (CTLs) and memory T-cytotoxic lymphocytes, which are responsible for degradation of cells and which bear the respective antigens.

Besides the hemopoietic stem cells, mesenchymal stem cells and mesenchymal adult progenitor cells are present in the bone marrow. All these stem cells appear to be capable of trans-differentiating into hepatocytes.

Thus, when using hemopoietic stem cells for introduction of a nucleotide construct according to the invention, all of the cells that are derived from the stem cell will bear the nucleotide construct in their genome and these cells will cause a blockade in the HIV replication and in the further spread of HIV particles. Also, they will not be killed by virus particle outburst and thus no AIDS symptoms will emerge. With an average lifetime of 100 days for a mature white blood cell, it will be easy to see that within a relatively short period of time, all of the infected and unprotected cells will disappear from the bloodstream, while they are replaced by cells that have been formed from the transgenic stem cells. If, in these transgenic cells, a provirus becomes active and starts to produce the gene products from transcription of the proviral DNA, the nucleotide construct of the invention, which is able to produce one transcript or multiple transcripts capable to fold into dsRNA, will induce an intracellular and cytoplasmic mechanism in which homologous viral RNA molecules are specifically degraded in a process referred to as RNA silencing. As a consequence, the expression of one or multiple viral genes essential for virus replication will be inhibited and no reproduction of virus particles and an eventual outburst of particles will occur anymore. Furthermore, the presence of the nucleotide construct of the invention in these cells does not impair the normal function of the cell.

For treatment of KSHV and EBV that both occur in their latent forms in B-lymphocytes, a nucleotide construct that expresses a dsRNA and is able to inhibit replication of the viruses is introduced into pluripotent hemopoietic stem cells.

Hepatitis C virus infects and becomes resident in cells of the liver. In addition, HCV appears to also reside in a latent state in specific blood cells. For HCV, the target cells represent hepatocytes. Progenitor cells of these hepatocytes are pluripotent liver stem cells or, alternatively, mesenchymal stem cells, mesenchymal adult progenitor cells, or hemopoietic stem cells. It is, of course, possible to introduce a nucleotide construct in these progenitor cells in a similar way as described herein for the hemopoietic stem cells. When using progenitor cells for introduction of a nucleotide construct according to the invention and after introduction of the cells into the liver of a person, all of the hepatocytes that are derived from the progenitor cells will bear the nucleotide construct in their genome and these cells will cause a blockade in the HCV replication and in further spread of HCV particles.

Transduction of human hemopoietic stem cells is known in the art. Chatterjee et al. (Blood 93:1882-1894, 1999) describe a system for transduction of human myeloid progenitor cells using AAV. AAV is a single-stranded, replication-defective nonpathogenic human parvovirus with a 4.7 kb DNA genome with palindromic inverted repeats. Wild-type AAV requires co-infection with a helper virus, such as, adenovirus or herpes simplex virus, for successful infection. In the absence of helper virus co-infection, AAV integrates via the inverted repeats into the AAVS1 site of human chromosome 19. AAV vectors lacking the P5 promoter region integrate into other chromosomal sites than AAVS1. AAV vectors allow efficient transgene expression from RNA polymerase II- and III-dependent promoters. The results from Chatterjee et al. show that primitive myelo-erythroid progenitor cells are amenable to genetic modification by AAV vectors.

Besides AAV, lentiviral vectors and, to a lesser extent, retroviral vectors, are efficient at transducing hemopoietic stem cells (Logan et al., Current Opinion in Biotechnology 2002, 13:429-436).

The target gene(s) of the virus that are subject to inhibition by the nucleotide construct of the invention are preferably genes that are essential for replication of the virus. In the case of HIV, such genes are the tat, rev and nef genes. The tat, rev and nef regulatory genes, from which tat and rev have overlapping coding domains, are essential for provirus activation and accumulation of viral structural proteins and genomic RNAs. The tat, rev and nef genes are encoded by the early multiple-spliced mRNA molecules. The sequences in the nucleotide construct can be derived from a single HIV gene or from multiple HIV genes. In the case of HCV, which only has a single gene, the target sequence can be any part of the viral genome.

To achieve production of a single transcript or multiple transcripts capable of forming a dsRNA, a nucleotide DNA construct, also termed the transgene cassette, is prepared that comprises both a sense and an antisense nucleotide sequence in opposite polarities (i.e., so that the antisense sequence is the reverse complement of the sense sequence), wherein both sense and antisense sequences have a length of at least 40 nucleotides. Further, in order to provide for a transcript that effectively inhibits gene expression in a target cell, either the sense or antisense strand of the transgene cassette is complementary or exhibits nucleotide sequence homology to the gene of the target cell, preferably to a target viral gene. A length of 40 nucleotides has been described to be minimally required for a dsRNA to effectively block target gene expression. The DNA construct that is to be stably integrated into the target cell genome and from which the gene silencing dsRNA is transcribed should, therefore, comprise at least about 80 nucleotides. Preferably larger stretches of DNA, with a length of 100, 150, 200, 250, 300, 350, 400, 450, or 500 nucleotides should be used in the transgene cassette.

The RNA transcribed from the nucleotide construct that is stably integrated in the host cell genome should form into a dsRNA or RNA duplex. Suitably, therefore, the RNA comprises inverted repeats or is of a palindromic structure but preferably comprises antisense and sense sequences. The dsRNA may be in the form of a hairpin structure or panhandle folding, whereby the sense and antisense regions are separated by introns or lariat structures (Moore et al. 1993, The RNA World, eds. Gesteland, R. F. and Atkins, J. F., Cold Spring Harbor Lab. Press, Plainview, N.Y., pp. 303-357). Preferably, the two RNA strands that form the RNA duplex are held together by a spacer region that forms a loop. More preferably, the two RNA strands that form the RNA duplex are held together by a spacer region that is left when the intron is spliced out. The sense and antisense nucleotide sequences can be connected through a spacer nucleotide sequence of any length (such as an intron sequence). If an intact intron sequence is used as spacer, this intron may be removed during mRNA formation by endogenous processes in the nucleus of the cell. The order of the sense and antisense sequence is not essential, although an antisense-sense orientation is preferred for reasons that such a transcript is unlikely to code for a protein. It is also possible to combine more than one sense-antisense combination in one and the same construct. The simple form can be depicted as: prom-AS-spac-S-term, wherein prom represents a promoter, S the target viral DNA sequence, AS the target viral DNA sequence in opposite polarity compared to S, “spac” a spacer sequence and “term” the transcriptional terminator DNA sequence. Also, the following constructs can be applied: prom-AS1-spac-S1-spac-AS2-spac-S2-term, or prom-AS2-spac-AS1-spac-S1-spac-S2-term. Variations in the composition of the construct are possible, as long as the transcripts of the constructs may fold partially or fully into dsRNA.

Alternatively, the nucleotide construct may consist of two separate transgene cassettes in which the target viral DNA sequence is present in opposite polarities towards the promoter(s). In short notation, these constructs then look like: prom1-S-term1 and prom2-AS-term2. Prom1 and prom2 can be the same or different promoters; term1 and term2 can be the same or different terminators. Both constructs can be introduced into the cell on the same vector, but can also be introduced using two different vectors.

Alternatively, the nucleotide construct may consist of a single target viral DNA sequence flanked by promoters in opposite polarities in terms of transcriptional directions. In short notation, such a construct then is formed by: prom1-S-prom2. Prom1 and prom2 can be the same or different promoters.

The transcriptional promoter is preferably derived from DNA viruses, including 5′-long terminal repeats from retroviruses and lentiviruses, the SV40 large T-antigen promoter, the cytomegalus virus (CMV) immediate early promoter, and the like. These promoters and various others are easily obtainable for a person skilled in the art. As terminator, any terminator applicable in human or animal cells can be used.

For an effective anti-HIV therapy, basically, the following steps should be taken:

1. Isolation of Stem Cells

In the case of isolation of human hemopoietic stem cells from HIV-infected persons, bone marrow punctions will be taken from HIV seropositive patients. These punctions generally harbor 10⁸ cells per milliliter, 50,000 of which are hemopoietic stem cells. Using standard enrichment techniques, an amount of 5×10⁵ CD34⁺38⁻ cells will be isolated per milliliter of punction and, in principle, one out of ten of these cells is a HSC.

2a. Construction of the Transgenic Cassette

According to generally used genetic engineering techniques, a nucleotide construct is made which comprises one or more promoters and terminators to enable transcription and formation of dsRNA as described above.

2b. Transduction of Stem Cells

The cell fractions enriched for hemopoietic stem cells and lymphoid stem cells will preferably be transduced using recombination. For this, the nucleotide constructs as prepared according to the description above are preferably inserted in a viral vector for stable integration into the target cell chromosome. Transfection or transduction of stem cells is done using methods known in the art (e.g., Halene and Kohn, Human Gene Therapy 11:1259-1267, 2000; Buchsacher and Wong-Stall, Blood 95:2499-2504, 2000; Buchsacher and Wong-Staal, Human Gene Therapy 12:1013-1019, 2001).

3. Reintroduction

The transduced HIV-resistant hemopoietic stem cells and lymphoid stem cells are then re-introduced into the bone marrow of the patient and a new HIV-resistant T-helper cell population will be established protecting the patient from AIDS symptom development and rendering the patient non-infectious.

A person skilled in the art will be able to use the invention in similar set-ups for treatment of infections with other chronic viruses.

The present invention is not only suitable for the treatment of animals or humans who are already infected by a chronic virus, but may also be used as a prophylactic to inhibit infection with such a virus. This would be especially recommendable for people who run a high risk of being infected with a chronic virus.

The invention is further explained with the aid of the following illustrative examples.

### EXAMPLES

### Example 1

### Molecular Cloning of the, HIV-1 Rev, Tat and Nef Genes

Purified DNA of an infectious cDNA clone of HIV-1, denoted pLai (Peden K., et al., 1991, Virology 185:661-672), was used as a template for cloning of the HIV-1 Rev, Tat and Nef genes, using DNA-based PCR. Four oligonucleotides were designed WdV001: GCGGCCGCATGGCAGGAAGAAGCGGAG (SEQ ID NO:_) and WdV002: GAGGTGGGTTGCTTT GATAGAGAAACTTGATG (SEQ ID NO:_), flanking the first Rev exon and WdV003: CAAAGCAACCCACCTCCCAACCCCGAG (SEQ ID NO:_) and WdV004: GCGGCCGCTATTCTTT AGTTCCTGACTCC (SEQ ID NO:_), flanking the second Rev exon. Oligonucleotides WdV001 and WdV004 contain a NotI recognition site, which is absent in the Lai Rev DNA sequence. Purified Lai DNA was subjected to PCR using oligonucleotides WdV001 and WdV002 and oligonucleotides WdV003 and WdV004, yielding the Rev exon1 and exon2 cDNA molecules, respectively. Both DNA fragments were agarose gel purified, mixed and subjected to a second round of PCR amplification using oligonucleotides WdV001 and WdV004. The 366 base pairs-long DNA fragment was directly ligated into the pGEM-T vector (Promega), yielding the recombinant plasmid pGEM-T-Rev.

Similarly, four oligonucleotides were designed WdV005: GCGGCCGCATGGCAGCCAGTAGATCCTAGAC (SEQ ID NO:_) and WdV006: GAGGTGGGTTGCTTTGATAGAGAAACTTGATG (SEQ ID NO:_), flanking the first Tat exon and WdV007: CTATCAAAGCAACCCACCTCCCAACCCCGAGG (SEQ ID NO:_) and WdV008: GCGGCCGCTATTCTTTAGTTCCTGACTCC (SEQ ID NO:_), flanking the second Tat exon. Oligonucleotides WdV005 and WdV008 contain a NotI recognition site, which is absent in the Lai Tat DNA sequence. Purified Lai DNA was subjected to PCR using oligonucleotides WdV005 and WdV006 and oligonucleotides WdV007 and WdV008, yielding the Tat exon1 and exon2 cDNA molecules, respectively. Both DNA fragments were agarose gel purified, mixed and subjected to a second round of PCR amplification using oligonucleotides WdV005 and WdV008. The 276 base pairs-long DNA fragment was directly ligated into the pGEM-T vector (Promega), yielding the recombinant plasmid pGEM-T-Tat.

Two oligonucleotides were designed WdV011: ATAAGAATGCGGCCGCATGGGTGGCAAGTGGTCAAAAAGTAG (SEQ ID NO:_) and WdV012: ATAGTTTAGCGGCCGCTCAGCAGTTCTTGAAGTACTCCGGATG (SEQ ID NO:_), flanking the Nef coding sequence. Both oligonucleotides contain a NotI recognition site, which is absent in the Lai Nef DNA sequence. Purified Lai DNA was subjected to PCR using oligonucleotides WdV011 and WdV012, yielding the Nef cDNA molecule. The 653 base pairs-long DNA fragment was directly ligated into the pGEM-T vector (Promega), yielding the recombinant plasmid pGEM-T-Nef.

### Example 2

### Construction of the GFP Reporter DNA Constructs

The hrGFP coding sequence was generated by PCR using purified DNA of plasmid pFB-hrGFP (Stratagene) as a template, with oligonucleotides WdV020: TCACCATGGTGAGCAAGCAGATCCTG (SEQ ID NO:_) and WdV021: ATTACACCCACTCGTG CAGGCTGC (SEQ ID NO:_) flanking the hrGFP coding sequence. The amplified DNA fragment was cloned into pEF5/FRT/V5-DEST using “gateway” (Invitrogen) following the manufacturer's recommendations, yielding pWdV08 (sense, s-GFP).

A DNA fragment comprising the human EF1α promoter was generated by PCR using DNA of plasmid pEF5/FRT/V5-DEST as a template with oligonucleotides WdV034: ACATGCATGCTGGGGATGCGGTGGGCTCTATGGATGTCGCGTGAGGCTCCGGTGCCC GTCAG (SEQ ID NO:_) (with a SphI restriction site) and WdV035: CTCCATGGTGAATCACGA CACCTGAAATGGAAGAAAAAAACTTTG (SEQ ID NO:_). A 300 base pairs anti-sense hrGFP DNA fragment was generated by PCR using DNA of plasmid pWdV08 as a template and oligonucleotides; WdV036: GGTGTCGTGATTCACCATGGAGGGCTGCGGCAAGGGCAACATCCTG (SEQ ID NO:_) and WdV037: CGGGATCCCGAGGCCGGTGATGGTCTTCTTCATCACGGGGCCGTCG (SEQ ID NO:_) (with a BamHI restriction site). Both DNA fragments were mixed at an equimolar ratio and re-amplified by PCR using oligonucleotides WdV034 and WdV037. The DNA fragment was digested with SphI and BamHI, isolated from an agarose gel and cloned into likewise digested pUC19 (Stratagene), yielding pWdV09.

A DNA fragment comprising the poly-adenylation signal of SV40 was generated by PCR using DNA of pPUR (Clontech) as a template with oligonucleotides WdV040: CCCTCCATGGTGAATTTACTTGCTTTAAAAAACCTCCCAC (SEQ ID NO:_) and WdV041: GGAATTCACATGCATGCTGGGGATGCGGTGGGCTCTATGGTGCAGAC ATGATAAGATACATTGATGAGTTTGGAC (SEQ ID NO:_) (containing EcoRI and SphI restriction sites). A 300 base pairs sense hrGFP DNA fragment was generated by PCR using DNA of plasmid pWdV08 as a template and oligonucleotides; WdV038: CGGGATCCGCCCTTGTAC TCCACGCGGTACACGAACATCTC (SEQ ID NO:_) (with a BamHI restriction site) and WdV039: GCAAGTAAATTCACCATGGAGGGCTGCGGCAAGGGCA (SEQ ID NO:_). Both DNA fragments were mixed at an equimolar ratio and re-amplified by PCR using oligonucleotides WdV038 and WdV040. The DNA fragment was digested with BamHI and EcoRI, isolated from an agarose gel and cloned into likewise digested pWdV09, yielding pWdV10 (antisense-stuffer-sense, ass-GFP).

### Example 3

### Construction of Sense Plus Antisense DNA Constructs Comprising the HIV-1 Tat, Rev and Nef Gene Sequences

Construction of antisense-stuffer-sense (ass) Vectors.

1) Construction of the ass-Tat Construct

The EF1α promoter, the GATEWAY cassette and BGH poly-adenylation signal of plasmid pEF5/FRT/V5-DEST (Invitrogen) were removed by digesting the plasmid with HindIII and SphI. A multilinker DNA fragment, containing HindIII, NotI, SacI, AvrII, XhoI, SgrAI, PacI and SphI restriction sites was generated by PCR using oligonucleotides;

WdV022:

(SEQ ID NO:_)

CCCAAGCTTGCGTAGAACCTGCGGCCGCTAATCTCGTGCGAG,
WdV023:

(SEQ ID NO:_)

GCAAGGCCTAGGCGATGATAGTTATGAGAGCTCGCACGAGA
TTAGCGGCC,
WdV024:

(SEQ ID NO:_)

CGCCTAGGCCTTGCTTCGCTCGAGCATCTGATTCGCCGGTGATCCG
and
WdV025:

(SEQ ID NO:_)

AGCTACAAGCATGCACGATACAGTTAATTAAAACGGATCACC
GGCGA ATC.

The resulting DNA fragment is digested with HindIII and SphI, isolated from an agarose gel and cloned into likewise digested pEF5/FRT/V5-DEST, yielding pWdV06.3 (empty expression vector).

A DNA fragment comprising the human EF1α promoter was generated by PCR using DNA of plasmid pEF5/FRT/V5-DEST as a template with oligonucleotides WdV047: CCCAAGCTTGGGCGTGAGGCTCCGGTGC (with a HindIII restriction site) (SEQ ID NO:_) and WdV048: AGGCCCGAAGGAATATCACGACACCTGAAATG (SEQ ID NO:_).

A 300 base pairs anti-sense Tat DNA fragment was generated by PCR using DNA of plasmid pGEM-T-Tat as a template and oligonucleotides; WdV049: AGGTGTCGTGATATTCCTTCGGGCCTGTCG (SEQ ID NO:_) and WdV050: AATAGTTTA GCGGCCGCAGATCCTAGACTAGAGC (with a NotI restriction site) (SEQ ID NO:_). Both DNA fragments were mixed at an equimolar ratio and re-amplified by PCR using oligonucleotides WdV047 and WdV050. The DNA fragment was digested with HindIII and NotI, isolated from an agarose gel and cloned into likewise digested pWdV06.3, yielding pWdV16.

A DNA fragment comprising the poly-adenylation signal of the bovine growth hormone gene was generated by PCR using DNA of pEF5/FRT/V5-DEST as a template with oligonucleotides WdV053: CCGCGGCCGCGAAGGAATACTGTGCCTTCTAGTTGC CAGC (containing a NotI restriction site) (SEQ ID NO:_) and WdV054: CCCCTAGGCCATAG AGCCCACCGCATC (containing an AvrII restriction site) (SEQ ID NO:_). The DNA fragment was digested with AvrII and NotI, isolated from an agarose gel and cloned into likewise digested pWdV16, yielding pWdV17 (as-Tat).

A DNA fragment comprising the poly-adenylation signal of the bovine growth hormone gene was generated by PCR using DNA of pEF5/FRT/V5-DEST as a template with oligonucleotides WdV053: CGAAGGAATACTGTGCCTTCTAGTTGCCAGC (SEQ ID NO:_) and WdV054: CCTTAATTAACCATAGAGCCCACCGCATC (containing a PacI restriction site) (SEQ ID NO:_).

A 300 base pairs sense Tat DNA fragment was generated by PCR using DNA of plasmid pGEM-T-Tat as a template and oligonucleotides; WdV051: GTCCTAGGAGATCCTAGACTAGAGCCCTG (containing an AvrII restriction site) (SEQ ID NO:_) and WdV52: AACTAGAAGGCACAGTATTCCTTCGGGCCTG (SEQ ID NO:_). Both DNA fragments were mixed and re-amplified by PCR using oligonucleotides WdV051 and WdV054. The DNA fragment was digested with AvrII and PacI, isolated from an agarose gel and cloned into likewise digested pWdV16, yielding pWdV18 (ass-Tat).

2) Construction of the ass-Rev Construct

A DNA fragment comprising the human eF1α promoter was generated by PCR using DNA of plasmid pEF5/FRT/V5-DEST as a template with oligonucleotides WdV047: CCCAAGCTTGGGCGTGAGGCTCCGGTGC (containing a HindIII restriction site) (SEQ ID NO:_) and WdV055: TCAAATATTGGTGTCACGACACCTGAAATG (SEQ ID NO:_).

A 300 base pairs anti-sense Rev DNA fragment was generated by PCR using DNA of plasmid pGEM-T-Rev as a template and oligonucleotides; WdV056: AGGTGTCGTGACACCAATATTTGAGGGCTTC (SEQ ID NO:_) and WdV057: ATAGTTTA GCGGCCGCAGCGGAGACAGCGACGAAGACCTC (containing a NotI restriction site) (SEQ ID NO:_). Both DNA fragments were mixed at an equimolar ratio and re-amplified by PCR using oligonucleotides WdV047 and WdV057. The DNA fragment was digested with HindIII and NotI, isolated from an agarose gel and cloned into likewise digested pWdV06.3, yielding pWdV19.

A DNA fragment comprising the poly-adenylation signal of the bovine growth hormone gene was generated by PCR using DNA of pEF5/FRT/V5-DEST as a template with oligonucleotides WdV054: CCTTAATTAACCATAGAGCCCACCGCATC (containing a NotI restriction site) (SEQ ID NO:_) and WdV60: CTCAAATATTGGTGCTGTGCCTTCTAG. A 300 base pairs sense Rev DNA fragment was generated by PCR using DNA of plasmid pGEM-T-Rev as a template and oligonucleotides; WdV058: AGCCTAGGAGCGGAGACAGCGACGAAGAC (containing an AvrII restriction site) (SEQ ID NO:_) and WdV059: GAAGGCACAGCACCAATATT TGAGGGCTTC (SEQ ID NO:_). Both DNA fragments were mixed at an equimolar ratio and re-amplified by PCR using oligonucleotides WdV054 and WdV058. The DNA fragment was digested with AvrII and PacI, isolated from an agarose gel and cloned into likewise digested pWdV19, yielding pWdV20

3) Construction of the ass.Nef Construct.

A DNA fragment comprising the human eF1α promoter was generated by PCR using DNA of plasmid pEF5/FRT/V5-DEST as a template with oligonucleotides WdV047: CCCAAGCTTGGGCGTGAGGCTCCGGTGC (containing a HindIII restriction site) (SEQ ID NO:_) and WdV061: GATGGTGCTACTCACGACACCTGAAATGGAAG (SEQ ID NO:_). A 300 base pairs anti-sense Nef DNA fragment was generated by PCR using DNA of plasmid pGEM-T-Nef as a template and oligonucleotides; WdV062: CAGGTGTCGTGAGTAGCACCATCCAAAGG (SEQ ID NO:_) and WdV063: ATAGTTTAGCGGCCGCACAAGTAGCAATACAGCAGCTACC (containing a NotI restriction site) (SEQ ID NO:_). Both DNA fragments were mixed and re-amplified by PCR using oligonucleotides WdV047 and WdV063. The DNA fragment was digested with HindIII and NotI, isolated from an agarose gel and cloned into likewise digested pWdV06.3, yielding pWdV21.

A DNA fragment comprising the poly-adenylation signal of the bovine growth hormone gene was generated by PCR using DNA of plasmid pEF5/FRT/V5-DEST as a template with oligonucleotides WdV054: CCTTAATTAACCATAGAGCCCACCGCATC (SEQ ID NO:_) (containing a PacI restriction site) and WdV066: GATGGTGCTACCTGTGCCTTCTAGT TGCCAGC (SEQ ID NO:_).

A 300 base pairs sense Nef DNA fragment was generated by PCR using DNA of plasmid pGEM-T-Nef as a template and oligonucleotides; WdV064: AGCCTAGGACAAGTAGCAATACAGCAGCTAC (containing an AvrII restriction site) (SEQ ID NO:_), WdV065: AGAAGGCACAGGTAGCACCATCCAAAGGTC (SEQ ID NO:_).

Both DNA fragments were mixed at an equimolar ratio and re-amplified by PCR using oligonucleotides WdV054 and WdV064. The DNA fragment was digested with AvrII and PacI, isolated from an agarose gel and cloned into likewise digested pWdV21, yielding pWdV22.

Construction of the antisense-intron-sense (ais) Vectors

A 168 base pairs DNA fragment comprising a chimaeric intron fragment composed of the 5′-donor splice site of human β-globin intron 1 and the branch and 3′-acceptor splice site from an intron derived from the human heavy chain variable region of an immunoglobulin gene was generated by PCR with DNA of plasmid pRL-null (Promega) as a template and using two oligonucleotides with NotI and AvrII restriction sites: WdV077: CGATCGATCGAGCGGCCGCCAGGTAAGTATCAAGGTTACAAGACAG (containing a NotI site) (SEQ ID NO:_) and WdV078: AGCATACCTAGGCCTGTGGAGAGAAAGGCAAAGTG (containing an AvrII site) (SEQ ID NO:_). The DNA fragment was digested with NotI and AvrII, isolated from an agarose gel and cloned into likewise digested pWdV18, pWdV20 or pWdV22, yielding pWdV35 (ais-Tat), pWdV36 (ais-Rev) and pWdV37 (ais-Nef), respectively.

### Example 4

### Transient Expression of HIV-1-Derived Sense Plus Antisense DNA Constructs in SupT1 and C33A Cells Inhibits HIV-1 Replication

SupT1 human non-Hodgkin's T-lymphoma cells (Smith S. D., et al., 1984, Cancer Research 44:5657) are grown at 37° C. in RPMI 1640 medium with 2 mmol/L L-Glutamine (Gibco) supplemented with 10% Fetal bovine serum (Biochrom KG) and 100 μg/ml Zeocine (Invitrogen), 100 U/ml penicillin and 100 μg/ml streptomycin (RPMI medium). Twice a week, the cell cultures containing two times 10⁶ cells/ml are diluted ten times in RPMI medium and sub-cultured at 37° C.

Three ml of a SupT1 cell culture containing 10⁶ cells/ml is electroporated with 1 μg of pLai DNA and incubated at 37° C. Seven days post-electroporation, the amount of virus present in the supernatant is quantified using a p24 (capsid) enzyme-linked immunosorbent assay (ELISA) according to the manufacturer's protocol (Beckman-Coulter) and aliquots are stored at −70° C. for use as inoculum (SupT1-adapted Lai).

Three ml of SupT1 cell cultures containing 10⁶ cells/ml are electroporated with 4 μgs of plasmid DNA and incubated at 37° C. (Beerens, N. and Berkhout, B., 2002, Journal of Virology 76:2329-2339). One day post-electroporation, the cell cultures are inoculated with 0.2 μl of SupT1-adapted Lai and the syncytium formation is monitored daily up to 14 days post-infection. Syncytia are formed six days post-infection in the cultures electroporated with the control plasmids: empty expression vector, sGFP, ass-GFP and as-Tat. The numbers of syncytia formed in the cell cultures electroporated with the anti-HIV plasmids, ass-Tat, ass-Rev, ass-Nef, ais-Tat, ais-Rev and ais-Nef are much lower than those electroporated with the control plasmids.

C33A cervix carcinoma cells (Auersperg N., 1964. J. Natl. Cancer Inst. 32:135-163) are grown at 37° C. in Dulbecco's modified Eagle medium with 0.11 g/L sodium pyridoxine, MEM non-essential amino acids (Gibco), supplemented with 10% Fetal bovine serum (Biochrom KG) and 100 μg/ml Zeocine (Invitrogen), 100 U/ml penicillin and 100 μg/ml streptomycin (DME medium). Twice a week, the confluent cell cultures are diluted ten times in DME medium and sub-cultured at 37° C.

Three ml of a confluent C33A cell culture is co-transfected with 1 μg of pLai DNA using the calcium phosphate method (Das, A. T, Klaver, B., Klasens, B. I. F., van Wamel, L. B. and Berkhout, B. 1997, Journal of Virology 71:2346-2356) and incubated at 37° C. Seven days post-transfection, the amount of virus in the supernatant is quantified using p24 ELISA and aliquots are stored at −70° C. for use as inoculum (C33A-adapted Lai).

Three ml of confluent C33A cell cultures are co-transfected with 1 μg of plasmid DNA and 1 μg of pLai DNA using the calcium phosphate method and incubated at 37° C. Three days post-transfection, the amount of virus in the supernatants is quantified using p24 ELISA. The amounts of virus in the supernatant of cells transfected with the anti-HIV plasmids ass-Tat, ass-Rev, ass-Nef, ais-Tat, ais-Rev and ais-Nef are much lower than those in the supernatant of cells transfected with the control plasmids, empty expression vector, sGFP, ass-GFP and as-Tat.

### Example 5

### Production of 293 and Jurkat Cell Lines Stably Expressing HIV-1-Derived Sense Plus Antisense Gene Sequences

Flp-In 293 human embryonic kidney cells (Graham et al., 1977, J. Gen. Virol. 36:59-74; purchased from Invitrogen) harboring an FRT recombination site are grown at 37° C. in DME medium. Twice a week the confluent cell cultures are diluted ten times in DME medium and sub-cultured at 37° C.

Flp-In Jurkat human T-cell leukemia cells (Weiss et al., 1984, J. Immunol. 133:123-128; purchased from Invitrogen) harboring an FRT recombination site are grown at 37° C. in RPMI medium. Twice a week, the cell cultures containing two times 10⁶ cells/ml are diluted ten times in RPMI medium and sub-cultured at 37° C.

Plasmid DNA is recombined into the FRT recombination sites of both cell lines according to the manufacturer's recommendations (Invitrogen). Cell batches with plasmid DNA stably integrated into their FRT recombination sites on the chromosomal DNA are grown and selected in appropriate medium provided with 100 μg/ml hygromycin.

### Example 6

### Flp-In 293 Cells Stably Expressing HIV-1-Derived Sense Plus Antisense Gene Sequences are Resistant Against HIV-1 Infection

Three mls of confluent Hygromycin-resistant and Zeocine-sensitive Flp-In 293 cell cultures with integrated plasmids are transfected with 1 μg of pLai DNA using lipofectamine according to the manufacturer's recommendations (Invitrogen) and incubated at 37° C. Seven days post-infection, the amount of virus in the supernatants is quantified using p24 ELISA. The amounts of virus in the supernatant of cells harboring the anti-HIV plasmids, ass-Tat, ass-Rev, ass-Nef, ais-Tat, ais-Rev and ais-Nef are much lower than those in the supernatant of cells harboring the control plasmids, empty expression vector, sGFP, ass-GFP and as-Tat.

### Example 7

### Flp-In Jurkat Cells Stably Expressing HIV-1-Derived Sense Plus Antisense Gene Sequences are Resistant Against HIV-1 Infection

C33A-adapted Lai or SupT1-adapted Lai are inoculated to Flp-In Jurkat cells and the infection process was monitored for 30 days post-infection using p24 ELISA.

C33A-adapted virus starts to replicate in Flp-In Jurkat cells 11 to 14 days post-infection. Virus particles in the supernatant of these cells are re-inoculated to Flp-In Jurkat cells. Virus replication starts seven days post-infection, yielding a Jurkat-adapted Lai strain, which is used in further infection experiments.

Three ml of Hygromycin-resistant and Zeocine-sensitive Flp-In Jurkat cell cultures with integrated plasmids are inoculated with 1 μl of Jurkat-adapted Lai and incubated at 37° C. Fourteen days post-infection, the amount of virus in the supernatants is quantified using p24 ELISA. The amounts of virus in the supernatant of cells harboring the anti-HIV plasmids, ass-Tat, ass-Rev, ass-Nef, ais-Tat, ais-Rev and ais-Nef are much lower than those in the supernatant of cells harboring the control plasmids, empty expression vector, sGFP, ass-GFP and as-Tat.

Fourteen days post-infection, 10 μl of each supernatant was inoculated to 3 mls of Jurkat cultures containing 10⁶ cells/ml and the amounts of virus in the supernatants is quantified using p24 ELISA seven days post-infection. The supernatants of the cell cultures harboring the anti-HIV constructs do not accumulate infectious virus particles in contrast to the cell cultures harboring the control plasmids.

1. A method for inhibiting replication of a chronic virus in cells of an animal or human, said method comprising:
stably integrating a nucleotide construct into a cell's genome, wherein said cell is a target cell of the chronic virus or a progenitor cell thereof capable of generating said target cell,
wherein the nucleotide construct is able to produce in said cell one transcript or multiple transcripts capable of forming a double-stranded RNA with nucleotide sequence homology to at least one nucleotide sequence of the chronic virus, said nucleotide sequence being essential for replication of the chronic virus.

2. A method for inhibiting replication of a chronic virus in cells of an animal or human, said method comprising:
stably integrating a nucleotide construct into a cell's genome, wherein said cell is a target cell of the chronic virus or a progenitor cell thereof capable of generating said target cell,
wherein the nucleotide construct produces in said cell one transcript or multiple transcripts that form a double-stranded RNA having nucleotide sequence homology to one or more nucleotide sequences of the chronic virus coding for one or more early viral genes.

3 The method according to claim 1, wherein said nucleotide construct is stably integrated into the cell's genome by transducing said cell with a vector of viral origin, said vector derived from a virus selected from the group consisting of retroviruses, lentiviruses, adenovirus, adeno-associated virus, herpes simplex virus, and adenovirus-AAV hybrid.

4. The method according to claim 1, wherein said stable integration is achieved into the AAVS1 site of human chromosome 19 by transducing said cell with a viral vector derived from an adeno-associated virus or adenovirus-AAV hybrid vector.

5 The method according to claim 1, wherein said cell is a stem cell.

6. A method of inhibiting human immunodeficiency virus replication in a person, said method comprising:
a) isolating, from the person, cells selected from the group consisting of hemopoietic stem cells, lymphoid stem cells, T-helper lymphocytes, and mixtures thereof;
b) stably integrating, into the isolated cells' genomes, a nucleotide construct able to produce, in said cells, at least one transcript capable of forming a double-stranded RNA having nucleotide sequence homology to at least one nucleotide sequence of human immunodeficiency virus essential for replication of human immunodeficiency virus; and
c) re-introducing said isolated cells into the person from whom they were isolated.

7. The method according to claim 6 wherein the at least one nucleotide sequence of human immunodeficiency virus essential for replication of human immunodeficiency virus is selected from the group consisting of HIV tat, rev, nef genes, and combinations thereof.

8. A method for inhibition of Hepatitis C virus replication in a person, said method comprising:
a) isolating hepatocytes, liver stem cells, mesenchymal adult progenitor cells, mesenchymal stem cells and/or hemopoietic stem cells from a person;
b) introducing, into said isolated cells, a nucleotide construct able to produce, in said isolated cells, at least one transcript capable of forming a double-stranded RNA with nucleotide sequence homology to at least one nucleotide sequences of the Hepatitis C virus; and
c) re-introducing said isolated cells into the person's liver.

9. A cell of animal or human origin, wherein said cell is a target cell for a chronic virus, said cell comprising:
in said cell's genome, a stably integrated nucleotide construct,
wherein said nucleotide construct is able to produce in said cell a single transcript or multiple transcripts capable of forming a double-stranded RNA with nucleotide sequence homology to one or more nucleotide sequences of said chronic virus which are essential for replication.

10. A progenitor cell of animal or human origin, which progenitor cell is able to generate a target cell for a chronic virus, said progenitor cell comprising:
an introduced nucleotide construct, which introduced nucleotide construct is able to produce a single transcript or multiple transcripts capable of forming a double-stranded RNA with nucleotide sequence homology to one or more nucleotide sequences of the chronic virus which are essential for replication,
wherein said introduced nucleotide construct is stably integrated into said progenitor cell's genome.

11. A cell selected from the group consisting of a human hemopoietic stem cell, human lymphoid stem cell, and human T-helper lymphocyte, said cell comprising:
a nucleotide construct introduced into said cell, which nucleotide construct is able to produce a double-stranded RNA which is homologous to one or more nucleotide sequences of the human immunodeficiency virus which are essential for replication,
wherein said nucleotide construct is stably integrated into the cell's genome.

12. A cell selected from the group consisting of a human hemopoietic stem cell, a human lymphoid stem cell, or a human T-helper lymphocyte, said cell comprising:
a nucleotide construct able to produce a double-stranded RNA homologous to one or more nucleotide sequences of the human immunodeficiency virus coding for tat, rev and/or nef in said cell,
wherein said nucleotide construct is stably integrated into the cell's genome.

13. A viral vector comprising:
a nucleotide construct harboring a nucleotide sequence of at least 40 nucleotides, said nucleotide sequence being homologous to at least part of a gene of a chronic virus capable of infecting a non-plant cell, wherein said nucleotide sequence is further
also present as an inverted repeat or
flanked by two promoters and
which nucleotide construct, when transcribed in a non-plant cell, yields at least one transcript capable of forming a double-stranded RNA from said nucleotide sequence and inverted repeat, or from said nucleotide sequence flanked by two promoters,
wherein said viral vector is further capable of stably integrating said nucleotide construct into the non-plant cell's genome.

14. The viral vector of claim 13, wherein the nucleotide sequence and inverted repeat are separated by an intron.

15. The viral vector of claim 13, further comprising:
multiple nucleotide sequences of at least 40 nucleotides in length having nucleotide sequence homology to different parts of a gene, to different genes, or to different gene parts of said chronic virus.

16. The viral vector of claim 13, wherein said viral vector is of retrovirus, lentivirus, or adeno-associated virus origin.

17. The viral vector of claim 13, wherein said gene is an early viral gene.

18. A method of inhibiting replication of a chronic virus in a non-plant cell said method comprising contacting said non-plant cell with the viral vector of claim 13.

19. A method of treating an infection of a chronic virus in cells of a subject suffering from said infection, said method comprising:
contacting a subject's cells or progenitor cells thereof capable of generating said cells, with a therapeutically effective amount of a reagent comprising a nucleotide construct that stably integrates a nucleotide construct into the cells' genomes, which nucleotide construct is able to produce one transcript or multiple transcripts capable of forming a double-stranded RNA with nucleotide sequence homology to at least one nucleotide sequence of said chronic virus essential for replication of said chronic virus.

20. The method according to claim 19, wherein said chronic virus is human immunodeficiency virus and wherein said cells are hemopoietic stem cells, lymphoid stem cells or T-helper lymphocytes.

21. The method according to claim 19, wherein said chronic virus is Hepatitis C virus and wherein said cells are hepatocytes, liver stein cells, mesenchymal adult progenitor cells, mesenchymal stem cells or hemopoietic stem cells.

22. The method according to claim 19, wherein said reagent comprises a viral vector comprising:
a nucleotide construct harboring a nucleotide sequence of at least 40 nucleotides, which nucleotide sequence is homologous to at least part of a gene of the chronic virus, wherein said nucleotide sequence is
also present as an inverted repeat or
is flanked by two promoters and
which nucleotide construct, when transcribed, yields one transcript or multiple transcripts capable of forming a double-stranded RNA from said nucleotide sequence and inverted repeat, or from said nucleotide sequence flanked by two promoters,
wherein said viral vector is further capable of stably integrating said nucleotide construct into the cell's genome.

23. The method according to claim 19, wherein prior to said contacting with said reagent, said cells are isolated from the subject, and wherein said cells are thereafter re-introduced into the subject.

24. A method of inhibiting replication of a chronic virus in a cell, said cell selected from the group consisting of a target cell of the chronic virus or a progenitor cell of the target cell, said method comprising:
stably integrating a nucleotide construct into the cell's genome,
wherein the stably integrated nucleotide construct produces in said cell at least one transcript that forms a double-stranded RNA having nucleotide sequence homology to at least one viral nucleotide sequence involved in the chronic virus's replication.

25. A method for inhibiting replication of a chronic virus in a non-plant cell, said method comprising:
stably integrating, into the non-plant cell's genome, means for producing in said non-plant cell, at least one transcript that, in said non-plant cell, forms double-stranded RNA having nucleotide sequence homology to at least one viral nucleotide sequence encoding at least one early viral gene of the chronic virus.


SEQUENCE LISTING (50 sequences)

SEQ ID NO: 1 - DNA, 27 bp, Artificial
  Oligonucleotide WdV001
gcggccgcatggcaggaagaagcggag

SEQ ID NO: 2 - DNA, 32 bp, Artificial
  Oligonucleotide WdV002
gaggtgggttgctttgatagagaaacttgatg

SEQ ID NO: 3 - DNA, 27 bp, Artificial
  Oligonucleotide WdV003
caaagcaacccacctcccaaccccgag

SEQ ID NO: 4 - DNA, 29 bp, Artificial
  Oligonucleotide WdV004
gcggccgctattctttagttcctgactcc

SEQ ID NO: 5 - DNA, 31 bp, Artificial
  Oligonucleotide WdV005
gcggccgcatggcagccagtagatcctagac

SEQ ID NO: 6 - DNA, 32 bp, Artificial; identical to SEQ ID NO: 2
  Oligonucleotide WdV006

SEQ ID NO: 7 - DNA, 32 bp, Artificial
  Oligonucleotide WdV007
ctatcaaagcaacccacctcccaaccccgagg

SEQ ID NO: 8 - DNA, 29 bp, Artificial
  Oligonucleotide WdV008
gcggccgctattctttagttcctgactcc

SEQ ID NO: 9 - DNA, 42 bp, Artificial
  Oligonucleotide WdV011
ataagaatgcggccgcatgggtggcaagtggtcaaaaagtag

SEQ ID NO: 10 - DNA, 43 bp, Artificial
  Oligonucleotide WdV012
atagtttagcggccgctcagcagttcttgaagtactccggatg

SEQ ID NO: 11 - DNA, 26 bp, Artificial
  Oligonucleotide WdV020
tcaccatggtgagcaagcagatcctg

SEQ ID NO: 12 - DNA, 24 bp, Artificial
  Oligonucleotide WdV021
attacacccactcgtgcaggctgc

SEQ ID NO: 13 - DNA, 62 bp, Artificial
  Oligonucleotide WdV034
acatgcatgctggggatgcggtgggctctatggatgtcgcgtgaggctccggtgcccgtcag

SEQ ID NO: 14 - DNA, 45 bp, Artificial
  Oligonucleotide WdV035
ctccatggtgaatcacgacacctgaaatggaagaaaaaaactttg

SEQ ID NO: 15 - DNA, 46 bp, Artificial
  Oligonucleotide WdV036
ggtgtcgtgattcaccatggagggctgcggcaagggcaacatcctg

SEQ ID NO: 16 - DNA, 46 bp, Artificial
  Oligonucleotide WdV037
cgggatcccgaggccggtgatggtcttcttcatcacggggccgtcg

SEQ ID NO: 17 - DNA, 40 bp, Artificial
  Oligonucleotide WdV040
ccctccatggtgaatttacttgctttaaaaaacctcccac

SEQ ID NO: 18 - DNA, 75 bp, Artificial
  Oligonucleotide WdV041
ggaattcacatgcatgctggggatgcggtgggctctatggtgcagacatgataagatacattgatgagtttggac

SEQ ID NO: 19 - DNA, 41 bp, Artificial
  Oligonucleotide WdV038
cgggatccgcccttgtactccacgcggtacacgaacatctc

SEQ ID NO: 20 - DNA, 37 bp, Artificial
  Oligonucleotide WdV039
gcaagtaaattcaccatggagggctgcggcaagggca

SEQ ID NO: 21 - DNA, 42 bp, Artificial
  Oligonucleotide WdV022
cccaagcttgcgtagaacctgcggccgctaatctcgtgcgag

SEQ ID NO: 22 - DNA, 50 bp, Artificial
  Oligonucleotide WdV023
gcaaggcctaggcgatgatagttatgagagctcgcacgagattagcggcc

SEQ ID NO: 23 - DNA, 46 bp, Artificial
  Oligonucleotide WdV024
cgcctaggccttgcttcgctcgagcatctgattcgccggtgatccg

SEQ ID NO: 24 - DNA, 50 bp, Artificial
  Oligonucleotide WdV025
agctacaagcatgcacgatacagttaattaaaacggatcaccggcgaatc

SEQ ID NO: 25 - DNA, 28 bp, Artificial
  Oligonucleotide WdV047
cccaagcttgggcgtgaggctccggtgc

SEQ ID NO: 26 - DNA, 32 bp, Artificial
  Oligonucleotide WdV048
aggcccgaaggaatatcacgacacctgaaatg

SEQ ID NO: 27 - DNA, 30 bp, Artificial
  Oligonucleotide WdV049
aggtgtcgtgatattccttcgggcctgtcg

SEQ ID NO: 28 - DNA, 34 bp, Artificial
  Oligonucleotide WdV050
aatagtttagcggccgcagatcctagactagagc

SEQ ID NO: 29 - DNA, 40 bp, Artificial
  Oligonucleotide WdV053
ccgcggccgcgaaggaatactgtgccttctagttgccagc

SEQ ID NO: 30 - DNA, 27 bp, Artificial
  Oligonucleotide WdV054
cccctaggccatagagcccaccgcatc

SEQ ID NO: 31 - DNA, 31 bp, Artificial
  Oligonucleotide WdV053 PacI
cgaaggaatactgtgccttctagttgccagc

SEQ ID NO: 32 - DNA, 29 bp, Artificial
  Oligonucleotide WdV054 PacI
ccttaattaaccatagagcccaccgcatc

SEQ ID NO: 33 - DNA, 29 bp, Artificial
  Oligonucleotide WdV051
gtcctaggagatcctagactagagccctg

SEQ ID NO: 34 - DNA, 31 bp, Artificial
  Oligonucleotide WdV52
aactagaaggcacagtattccttcgggcctg

SEQ ID NO: 35 - DNA, 30 bp, Artificial
  Oligonucleotide WdVO55
tcaaatattggtgtcacgacacctgaaatg

SEQ ID NO: 36 - DNA, 31 bp, Artificial
  Oligonucleotide WdV056
aggtgtcgtgacaccaatatttgagggcttc

SEQ ID NO: 37 - DNA, 40 bp, Artificial
  Oligonucleotide WdV057
atagtttagcggccgcagcggagacagcgacgaagacctc

SEQ ID NO: 38 - DNA, 29 bp, Artificial
  Oligonucleotide WdVO54 NotI
ccttaattaaccatagagcccaccgcatc

SEQ ID NO: 39 - DNA, 27 bp, Artificial
  Oligonucleotide WdV60
ctcaaatattggtgctgtgccttctag

SEQ ID NO: 40 - DNA, 29 bp, Artificial
  Oligonucleotide WdV058
agcctaggagcggagacagcgacgaagac

SEQ ID NO: 41 - DNA, 30 bp, Artificial
  Oligonucleotide WdV059
gaaggcacagcaccaatatttgagggcttc

SEQ ID NO: 42 - DNA, 28 bp, Artificial
  Oligonucleotide WdV047 HindIII
cccaagcttgggcgtgaggctccggtgc

SEQ ID NO: 43 - DNA, 32 bp, Artificial
  Oligonucleotide WdV061
gatggtgctactcacgacacctgaaatggaag

SEQ ID NO: 44 - DNA, 29 bp, Artificial
  Oligonucleotide WdV062
caggtgtcgtgagtagcaccatccaaagg

SEQ ID NO: 45 - DNA, 40 bp, Artificial
  Oligonucleotide WdV063
atagtttagcggccgcacaagtagcaatacagcagctacc

SEQ ID NO: 46 - DNA, 32 bp, Artificial
  Oligonucleotide WdV066
gatggtgctacctgtgccttctagttgccagc

SEQ ID NO: 47 - DNA, 31 bp, Artificial
  Oligonucleotide WdV064
agcctaggacaagtagcaatacagcagctac

SEQ ID NO: 48 - DNA, 30 bp, Artificial
  Oligonucleotide WdV065
agaaggcacaggtagcaccatccaaaggtc

SEQ ID NO: 49 - DNA, 46 bp, Artificial
  Oligonucleotide WdV077
cgatcgatcgagcggccgccaggtaagtatcaaggttacaagacag

SEQ ID NO: 50 - DNA, 35 bp, Artificial
  Oligonucleotide WdV078
agcatacctaggcctgtggagagaaaggcaaagtg